(19) United States
(12) Patent Application Publication
(10) Pub. No.: US 2026/0237502 A1
(43) Pub. Date: Aug. 13, 2026

(54) SYSTEM, METHOD, AND COMPUTER PROGREAM PRODUCT FOR MEDICAL DEVICE EVENT MANAGEMENT

(71) Applicant: Bayer HealthCare LLC, Whippany, NJ (US)

(72) Inventors: Lois MILLER, Cecil, PA (US); Arthur UBER, III, Pittsburgh, PA (US); David GRIFFITHS, Pittsburgh, PA (US); Barry SKIRBLE, Allison Park, PA (US); Sri SHRIRAM, Murrysville, PA (US); Linda VAN ROOSMALEN, Gibsonia, PA (US)

(21) Appl. No.: 19/154,589

(22) PCT Filed: Feb. 28, 2024
(86) PCT No.: PCT/US2024/017648

§ 371 Date: Aug. 7, 2025

Related U.S. Application Data
(60) Provisional application No. 63/487,865, filed on Mar. 1, 2023.

(51) Int. Cl. G16H 40/40; G06F 3/16; H04L 67/12

(52) U.S. Cl. CPC G16H 40/40; G06F 3/167; H04L 67/12

(57) ABSTRACT

Systems for artificial intelligence based event management may include at least one processor to receive data associated with an event involving a medical device via a user interface (UI) associated with the medical device; receive data associated with an identification characteristic of the medical device; generate an event message; wherein the event message comprises the data associated with the event involving the medical device and the data associated with the identification characteristic of the medical device; provide a response message via the UI associated with the medical device based on the data associated with the event involving the medical device; where the response message includes information based on an output of a generative and conversational artificial intelligence (AI) platform; determine a category of the event message; and perform an action based on the category of the event message. Methods and computer program products are also disclosed.

### CROSS-REFERENCE TO RELATED APPLICATION

This application claims priority to U.S. Provisional Patent Application No. 63/487,865, filed on Mar. 1, 2023, the disclosure of which is incorporated by reference herein in its entirety.

### BACKGROUND

### 1. Field

This disclosure relates generally to systems, methods, and/or products that are used in diagnostic and interventional medical imaging procedures and, in non-limiting embodiments, to systems, methods, and computer program products for event management with regard to medical devices, including artificial intelligence based event management.

### 2. Technical Considerations

Medical Device Reporting (MDR) may be a subject of regulation that requires an entity, such as a manufacturer, to monitor device performance, detect potential device-related safety issues, and/or contribute to benefit-risk assessments of medical devices and related products.

Mandatory reporters, such as manufacturers, medical device user facilities, and importers of medical devices, may be required to submit to a government regulatory body certain types of reports for adverse events and/or product problems about medical devices. In addition, government regulatory bodies may also encourage health care professionals, patients, caregivers and/or consumers to submit voluntary reports about serious adverse events that may be associated with a medical device, as well as use errors, product quality issues, and therapeutic failures.

In some instances, government regulatory bodies may require medical device manufacturers to analyze customer complaints within a short amount of time of when the customer complaints are received. Due to the nature of the complaints, some complaints may be reportable to regulatory bodies. Medical device reporting timelines vary globally and can range from 2 days for a serious public health threat and between 3-30 days for all other reportable incidents based on the severity of the incident. This timeline may require that customer complaint information arrives in a short time and that information associated with the customer complaint is as complete as possible and accurately reported to all involved parties, including a medical device manufacturer.

### SUMMARY

Accordingly, provided are systems, methods, and computer program products for event management with regard to medical devices, including artificial intelligence (AI) based event management. This may include the use of machine learning models, including deep learning models.

Further non-limiting embodiments or aspects are set forth in the following numbered clauses:
Clause 1: A system for processing information associated with an event involving a medical device, comprising: at least one processor configured to: receive data associated with an event involving a medical device via a user interface (UI) associated with the medical device; receive data associated with an identification characteristic of the medical device; generate an event message, wherein the event message comprises the data associated with the event involving the medical device and the data associated with the identification characteristic of the medical device; provide a response message via the UI associated with the medical device based on the data associated with the event involving the medical device, wherein the response message comprises information based on an output of a generative and conversational artificial intelligence (AI) platform; determine a category of the event message based on at least one of the data associated with the event involving the medical device, the data associated with the identification characteristic of the medical device, or any combination thereof; and perform an action based on the category of the event message.
Clause 2: The system of clause 1, wherein the generative and conversational AI platform comprises at least one of a generative AI model, a conversational AI model, or any combination thereof.
Clause 3: The system of clauses 1 or 2, wherein the at least one processor is configured to: generate the response message based on receiving the data associated with the event involving the medical device, wherein, when generating the response message, the at least one processor is configured to: provide the data associated with the event involving the medical device as an input to a generative AI model; determine an output of the generative AI model based on the input, wherein the output comprises information associated with a potential resolution of the event involving the medical device; wherein, when providing the response message via the UI associated with the medical device, the at least one processor is configured to: provide the information associated with the potential resolution of the event involving the medical device.
Clause 4: The system of any of clauses 1-3, wherein, when providing the response message via the UI associated with the medical device, the at least one processor is configured to: provide a prompt to receive information from a user via the UI associated with the medical device, wherein the prompt comprises a question associated with the event involving the medical device.
Clause 5: The system of any of clauses 1-4, wherein, when performing the action, the at least one processor is configured to: transmit the event message based on the category of the event message.
Clause 6: The system of any of clauses 1-5, wherein, when transmitting the event message, the at least one processor is configured to: transmit the event message to a healthcare provider communication network; transmit the event message to a system for trend analysis with regard to one or more events involving the medical device; transmit the event message to a communication gateway for further transmission; or any combination thereof.
Clause 7: The system of any of clauses 1-6, wherein the at least one processor is further configured to: capture data associated with a time stamp regarding the event involving the medical device, wherein the data associated with the time stamp comprises at least one of: data associated with a time at which the data associated with the event involving the medical device was received via the UI associated with the medical device; data associated with a time at which a malfunction of the medical device occurred; or any combination thereof.
Clause 8: The system of any of clauses 1-7, wherein the at least one processor is further configured to: provide the UI associated with the medical device on a mobile device.
Clause 9: The system of any of clauses 1-8, wherein, when providing the UI associated with the medical device on the mobile device, the at least one processor is configured to: provide the UI associated with the medical device on the mobile device via an application of the mobile device based on receiving an input prompt at the mobile device.
Clause 10: The system of any of clauses 1-9, wherein, when receiving the data associated with the event involving the medical device, the at least one processor is configured to: receive the data associated with the event involving the medical device via a chatbot that interacts with the UI associated with the medical device.
Clause 11: The system of any of clauses 1-10, wherein, when receiving the data associated with the event involving the medical device, the at least one processor is configured to: receive voice data from a user via the user interface (UI) associated with the medical device; convert the voice data to text data using a speech to text algorithm; and convert the text data to the data associated with the event involving the medical device using a natural language processing (NLP) algorithm.
Clause 12: A method for processing information associated with an event involving a medical device comprising: receiving, with at least one processor, data associated with an event involving a medical device via a user interface (UI) associated with the medical device; receiving, with the at least one processor, data associated with an identification characteristic of the medical device; generating, with the at least one processor, an event message, wherein the event message comprises the data associated with the event involving the medical device and the data associated with the identification characteristic of the medical device; providing, with the at least one processor, a response message via the UI associated with the medical device based on the data associated with the event involving the medical device, wherein the response message comprises information based on an output of a generative and conversational artificial intelligence (AI) platform; determining, with the at least one processor, a category of the event message based on at least one of the data associated with the event involving the medical device, the data associated with the identification characteristic of the medical device, or any combination thereof; and performing, with the at least one processor, an action based on the category of the event message.
Clause 13: The method of clause 12, wherein the generative and conversational AI platform comprises at least one of a generative AI model, a conversational AI model, or any combination thereof.
Clause 14: The method of clauses 12 or 13, further comprising: generating the response message based on receiving the data associated with the event involving the medical device, wherein generating the response message comprises: providing the data associated with the event involving the medical device as an input to a generative AI model; determining an output of the generative AI model based on the input, wherein the output comprises information associated with a potential resolution of the event involving the medical device; wherein providing the response message via the UI associated with the medical device comprises: providing the information associated with the potential resolution of the event involving the medical device.
Clause 15: The method of any of clauses 12-14, wherein providing the response message via the UI associated with the medical device comprises: providing a prompt to receive information from a user via the UI associated with the medical device, wherein the prompt comprises a question associated with the event involving the medical device.
Clause 16: The method of any of clauses 12-15, wherein performing the action comprises: transmitting the event message based on the category of the event message.
Clause 17: The method of any of clauses 12-16, wherein transmitting the event message comprises: transmitting the event message to a healthcare provider communication network; transmitting the event message to a system for trend analysis with regard to one or more events involving the medical device; transmitting the event message to a communication gateway for further transmission; or any combination thereof.
Clause 18: The method of any of clauses 12-17, further comprising: capturing data associated with a time stamp regarding the event involving the medical device, wherein data associated with the time stamp comprises at least one of: data associated with a time at which the data associated with the event involving the medical device was received via the UI associated with the medical device; data associated with a time at which a malfunction of the medical device occurred; or any combination thereof.
Clause 19: The method of any of clauses 12-18, further comprising: providing the UI associated with the medical device on a mobile device.
Clause 20: The method of any of clauses 12-19, wherein providing the UI associated with the medical device on the mobile device comprises: providing the UI associated with the medical device on the mobile device via an application of the mobile device based on receiving an input prompt at the mobile device.
Clause 21: The method of any of clauses 12-20, wherein receiving the data associated with the event involving the medical device comprises: receiving the data associated with the event involving the medical device via a chatbot that interacts with the UI associated with the medical device.
Clause 22: The method of any of clauses 12-21, wherein receiving the data associated with the event involving the medical device comprises: receiving voice data from a user via the user interface (UI) associated with the medical device; converting the voice data to text data using a speech to text algorithm; and converting to the text data to the data associated with the event involving the medical device using a natural language processing (NLP) algorithm.
Clause 23: A computer program product for processing information associated with an event involving a medical device, comprising at least one non-transitory computer-readable medium including one or more instructions, that when executed by at least one processor cause the at least one processor to: receive data associated with an event involving a medical device via a user interface (UI) associated with the medical device; receive data associated with an identification characteristic of the medical device; generate an event message, wherein the event message comprises the data associated with the event involving the medical device and the data associated with the identification characteristic of the medical device; provide a response message via the UI associated with the medical device based on the data associated with the event involving the medical device, wherein the response message comprises information based on an output of a generative and conversational artificial intelligence (AI) platform; determine a category of the event message based on at least one of the data associated with the event involving the medical device, the data associated with the identification characteristic of the medical device, or any combination thereof; and perform an action based on the category of the event message.

Clause 24: The computer program product of clause 23, wherein the generative and conversational AI platform comprises at least one of a generative AI model, a conversational AI model, or any combination thereof.

Clause 25: The computer program product of clauses 23 or 24, wherein the one or more instructions further cause the at least one processor to: generate the response message based on receiving the data associated with the event involving the medical device, wherein, the one or more instructions that cause the at least one processor to generate the response message cause the at least one processor to: provide the data associated with the event involving the medical device as an input to a generative AI model; determine an output of the generative AI model based on the input, wherein the output comprises information associated with a potential resolution of the event involving the medical device; wherein, the one or more instructions that cause the at least one processor to provide the response message via the UI associated with the medical device cause the at least one processor to: provide the information associated with the potential resolution of the event involving the medical device.

Clause 26: The computer program product of any of clauses 23-25, wherein, the one or more instructions that cause the at least one processor to provide the response message via the UI associated with the medical device cause the at least one processor to: provide a prompt to receive information from a user via the UI associated with the medical device, wherein the prompt comprises a question associated with the event involving the medical device.

Clause 27: The computer program product of any of clauses 23-26, wherein, the one or more instructions that cause the at least one processor to perform the action cause the at least one processor to: transmit the event message based on the category of the event message.

Clause 28: The computer program product of any of clauses 23-27, wherein, the one or more instructions that cause the at least one processor to transmit the event message cause the at least one processor to: transmit the event message to a healthcare provider communication network; transmit the event message to a system for trend analysis with regard to one or more events involving the medical device; transmit the event message to a communication gateway for further transmission; or any combination thereof.

Clause 29: The computer program product of any of clauses 23-28, wherein the one or more instructions further cause the at least one processor to: capture data associated with a time stamp regarding the event involving the medical device, wherein the data associated with the time stamp comprises at least one of: data associated with a time at which the data associated with the event involving the medical device was received via the UI associated with the medical device; data associated with a time at which a malfunction of the medical device occurred; or any combination thereof.

Clause 30: The computer program product of any of clauses 23-29, wherein the one or more instructions further cause the at least one processor to: provide the UI associated with the medical device on a mobile device.

Clause 31: The computer program product of any of clauses 23-30, wherein, the one or more instructions that cause the at least one processor to provide the UI associated with the medical device on the mobile device cause the at least one processor to: provide the UI associated with the medical device on the mobile device via an application of the mobile device based on receiving an input prompt at the mobile device.

Clause 32: The computer program product of any of clauses 23-31, wherein, the one or more instructions that cause the at least one processor to receive the data associated with the event involving the medical device cause the at least one processor to: receive the data associated with the event involving the medical device via a chatbot that interacts with the UI associated with the medical device.

Clause 33: The computer program product of any of clauses 23-32, wherein, when receiving the data associated with the event involving the medical device, the at least one processor is configured to: receive voice data from a user via the user interface (UI) associated with the medical device; convert the voice data to text data using a speech to text algorithm; and convert the text data to the data associated with the event involving the medical device using a natural language processing (NLP) algorithm.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the present disclosure. As used in the specification and the claims, the singular form of “a,” “an,” and “the” include plural referents unless the context clearly dictates otherwise.

### BRIEF DESCRIPTION OF THE DRAWINGS

Additional advantages and details of non-limiting embodiments or aspects are explained in greater detail below with reference to the exemplary embodiments that are illustrated in the accompanying schematic figures, in which:

FIG. 1 is a diagram of a non-limiting embodiment of an environment in which systems, methods, and/or products described herein, may be implemented, according to the principles of the present disclosure;

FIG. 2 is a diagram of a non-limiting embodiment of a system for providing AI based event management;

FIG. 3 is a diagram of a non-limiting embodiment of components of one or more systems or one or more devices of FIGS. 1 and 2;

FIG. 4 is a flowchart of a non-limiting embodiment of a process for processing information associated with an event involving a medical device; and

FIGS. 5A-5D are diagrams of an implementation of a non-limiting embodiment or aspect of a process for processing information associated with an event involving a medical device.

### DETAILED DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the present disclosure as it is oriented in the drawing figures. However, it is to be understood that the present disclosure may assume various alternative variations and step sequences, except where expressly specified to the contrary. It is also to be understood that the specific devices and processes illustrated in the attached drawings, and described in the following specification, are simply exemplary embodiments of the present disclosure. Hence, specific dimensions and other physical characteristics related to the embodiments or aspects of the embodiments disclosed herein are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise. The phrase “based on” may also mean “in response to” and be indicative of a condition for automatically triggering a specified operation of an electronic device (e.g., a processor, a computing device, etc.) as appropriately referred to herein.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and communicates the processed information to the second unit. In non-limiting embodiments, a message may refer to a network packet (e.g., a data packet and/or the like) that includes data. It will be appreciated that numerous other arrangements are possible.

As used herein, the term “system” may refer to one or more computing devices or combinations of computing devices such as, but not limited to, processors, servers, client devices, software applications, and/or other like components. In addition, reference to “a server” or “a processor,” as used herein, may refer to a previously-recited server and/or processor that is recited as performing a previous step or function, a different server and/or processor, and/or a combination of servers and/or processors. For example, as used in the specification and the claims, a first server and/or a first processor that is recited as performing a first step or function may refer to the same or different server and/or a processor recited as performing a second step or function.

Non-limiting embodiments of the present disclosure are directed to systems, methods, and computer program products for event management, including artificial intelligence (AI) based event management. In non-limiting embodiments or aspects, an event management system may receive data associated with an event involving a medical device via a user interface (UI) associated with the medical device, receive data associated with an identification characteristic of the medical device, generate an event message, where the event message comprises the data associated with the event involving the medical device and the data associated with the identification characteristic of the medical device, provide a response message via the UI associated with the medical device based on the data associated with the event involving the medical device, where the response message comprises information based on an output of a generative and conversational artificial intelligence (AI) platform, determine a category of the event message based on at least one of the data associated with the event involving the medical device, the data associated with the identification characteristic of the medical device, or any combination thereof, and perform an action based on the category of the event message. In non-limiting embodiments, the generative and conversational AI platform may include at least one of a generative AI model, a conversational AI model, such as a chatbot, or any combination thereof. In non-limiting embodiments, information (e.g., in the form of feedback) communicated to or received from a user via a UI may be via a visual display and/or via an auditory feedback such as for example sound or language. In some non-limiting embodiments, the medical device may include a fluid injector, a treatment device, such as a therapeutic device, including but not limited to a robotic surgical device, a radiation therapy device, and/or the like. Additionally, or alternatively, the medica device may include a diagnostic device.

In non-limiting embodiments, the event management system may generate the response message based on receiving the data associated with the event involving the medical device, and when generating the response message, the event management system may provide the data associated with the event involving the medical device as an input to a generative AI model, determine an output of the generative AI model based on the input, where the output comprises information associated with a potential resolution of the event involving the medical device. In non-limiting embodiments, when providing the response message via the UI associated with the medical device, the event management system may provide the information associated with the potential resolution of the event involving the medical device.

In non-limiting embodiments, when providing the response message via the UI associated with the medical device, the event management system may provide a prompt to receive information from a user via the UI associated with the medical device, wherein the prompt includes a question associated with the event involving the medical device. In non-limiting embodiments, when performing the action, the event management system may transmit the event message based on the category of the event message. In non-limiting embodiments, when transmitting the event message, the event management system may transmit the event message to a healthcare provider communication network, transmit the event message to a system for trend analysis with regard to one or more events involving the medical device, transmit the event message to a communication gateway for further transmission, or any combination thereof.

In non-limiting embodiments, the event management system may capture data associated with the medical device and/or data associated with a time stamp regarding the event involving the medical device, wherein data associated with the time stamp may include at least one of data associated with a time at which the data associated with the event involving the medical device was received via the UI associated with the medical device, data associated with a time at which a malfunction of the medical device occurred, or any combination thereof. The data associated with the medical device may include data associated with device identification (e.g., a serial number, a unit identifier, etc.), data associated with a device location, data associated with a device owner of record, and/or data associated with a current user identification. The data associated with the medical device may also include data associated with a device use history (e.g., a number of procedures, such as injections, etc.) and data associated with a device service history (e.g., an amount of time the device has been in service, etc.) such as disclosed in U.S. Pat. Nos. 10,522,250; 11,232,862, U.S. Patent Application Publication 2022/0375586A1; and U.S. Patent Application Publication 2023/0364325A1, all of which owned by Bayer HealthCare LLC and incorporated herein by reference.

In non-limiting embodiments, the event management system may provide the UI associated with the medical device on a mobile device. In non-limiting embodiments, when providing the UI associated with the medical device on the mobile device, the event management system may provide the UI associated with the medical device on the mobile device via an application of the mobile device based on receiving an input prompt by the mobile device. In non-limiting embodiments, when receiving the data associated with the event involving the medical device, the event management system may receive the data associated with the event involving the medical device via a chatbot that interacts with the UI associated with the medical device. In non-limiting embodiments, when receiving the data associated with the event involving the medical device, the event management system may receive voice data from a user via the UI associated with the medical device, convert the voice data to text data using a speech to text algorithm, and convert the text data to the data associated with the event involving the medical device using a natural language processing (NLP) algorithm.

In this way, non-limiting embodiments of the present disclosure provide an event management system that may accurately identify, record, and/or transmit information associated with an event involving a medical device within a predetermined amount of time from the event taking place. The event management system may include artificial intelligence, data base systems, and/or other computer programs as ways to implement the functions of the event management system. The event management system may utilize natural language and/or voice control that may be integrated into a radiology diagnostics setting. Further, according to some non-limiting embodiments, by integrating voice data in the form of natural speech into an event reporting process, the event management system may reduce time and increase accuracy of reporting details surrounding the event. Additionally, information collected may be converted into natural language (e.g., of a predetermined language) so that the event can be dealt with immediately by appropriate individuals. Additionally, information collected may be in the native language of the speaker or location in which the procedure is taking place, be made available to local authorities in such language, and/or be automatedly translated into a common language for central analysis and storage and further reporting as required. In non-limiting embodiments, the event management system may collect, report, and categorize adverse events and machine learning is applied to see trends between events, device type and event frequency. In non-limiting embodiments, the event management system may allow for automated reporting that can serve to connect with engineering, commercial, and/or service personnel (e.g., to send out service personnel and/or connect with a customer experiencing an issue). System improvements may be achieved through data analysis and time for both a technologist and the medical device manufacturer. Using natural language processing in the form of recording and/or translating of voice data (e.g., speech) may produce more accurate and timelier event information. In non-limiting embodiments, the event management system may automate communication information related to events, and data can be transmitted faster internally to the manufacturer to begin the event investigation with the most accurate details of an event. This proposed automated method of collecting, reporting, and/or categorization of events on the front line may replace existing manual processes that consume time and network resources involved in scheduling, calling, interviewing, and/or surveying individuals (e.g., technologists) while they work to serve other patients.

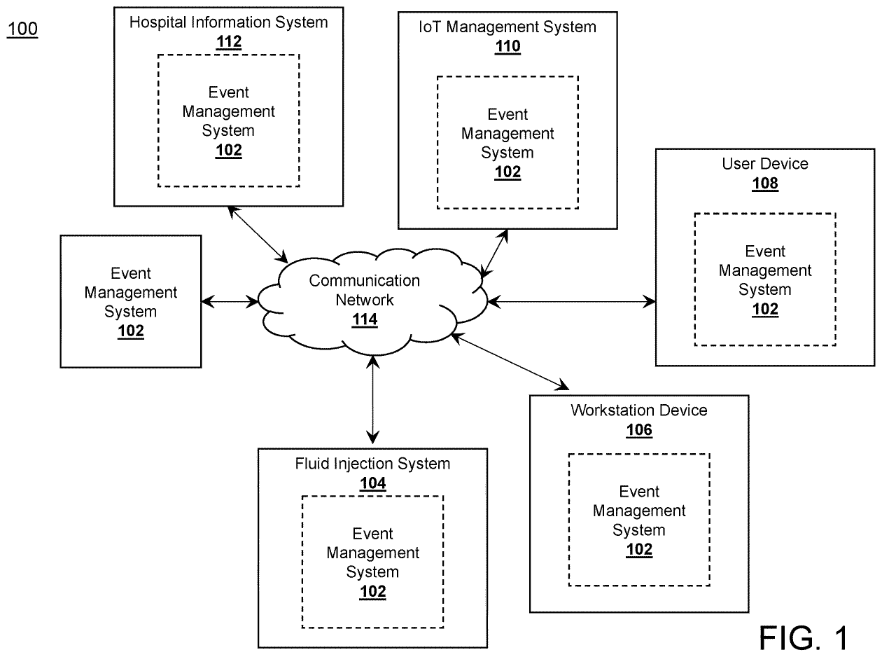

Referring now to FIG. 1, FIG. 1 is a diagram of a non-limiting embodiment of an environment 100 in which devices, systems, methods, and/or computer program products, described herein, may be implemented. As shown in FIG. 1, environment 100 includes event management system 102, fluid injection system 104, workstation device 106, user device 108, Internet of Things (IoT) management system 110, hospital information system 112, and communication network 114. In non-limiting embodiments, event management system 102, fluid injection system 104, workstation device 106, user device 108, IoT management system 110, and/or hospital information system 112 may interconnect (e.g., establish a connection to communicate) via wired connections, wireless connections, or a combination of wired and wireless connections. Any devices or systems in environment 100 may communicate with each other, either directly or indirectly, in a same or different communication network 114 as other devices or systems.

In non-limiting embodiments, event management system 102 may include one or more devices capable of being in communication with fluid injection system 104, workstation device 106, user device 108, IoT management system 110, and/or hospital information system 112 via communication network 114. For example, event management system 102 may include one or more computing devices, such as one or more computers, one or more servers (e.g., a cloud server, a group of servers, etc.), one or more desktop computers, one or more mobile devices (e.g., one or more tablets, one or more smartphones, etc.), and/or the like. In non-limiting embodiments, event management system 102 may include one or more (e.g., a plurality of) applications (e.g., software applications) that perform a set of functionalities on an external application programming interface (API) that allows event management system 102 to send data to an external system associated with the external API and to receive data from the external system associated with the external API. In non-limiting embodiments, the application may be supported by an application associated with user device 108 that would allow event management system 102, which may function as a workstation (e.g., a workstation of a control room for a site at which medical procedures are performed), to be the only one device that controls other systems and/or devices, and, in such an example, event management system 102 may provide an authentication function.

Additionally, or alternatively, event management system 102 may generate (e.g., train, validate, re-train, and/or the like), store, and/or implement (e.g., operate, provide inputs to and/or outputs from, and/or the like) one or more artificial intelligence (AI) models (e.g., machine learning models), such as a generative AI model. For example, event management system 102 may generate one or more machine learning models by fitting (e.g., validating) one or more AI models against data used for training (e.g., training data). In non-limiting embodiments or aspects, event management system 102 may generate, store, and/or implement one or more AI models that are provided for a real-time environment (e.g., a runtime environment) used for providing inferences based on data in a live situation. In non-limiting embodiments or aspects, event management system 102 may be in communication with a data storage device, which may be local or remote to event management system 102.

In non-limiting embodiments, event management system 102 may be capable of receiving (e.g., retrieving via a pull) information from, storing information in, transmitting information to, and/or searching information stored in a data source (e.g., a healthcare data source). In non-limiting embodiments, event management system 102 may be a component of fluid injection system 104, workstation device 106, user device 108, IoT management system 110, and/or hospital information system 112.

In non-limiting embodiments, event management system 102 may operate based on the use of a generative and conversational AI platform. For example, event management system 102 may include one or more AI algorithms (e.g., machine learning models) and/or one or more chatbots that may work in collaboration with each other to generate messages (e.g., response and/or event messages) associated with a medical device, such as a fluid injector, based on data associated with the event involving the medical device.

In non-limiting embodiments, fluid injection system 104 may include one or more devices capable of being in communication with event management system 102, workstation device 106, user device 108, IoT management system 110, and/or hospital information system 112 via communication network 114. For example, fluid injection system 104 may include one or more computing devices, such as one or more computers, one or more servers (e.g., a cloud server, a group of servers, etc.), one or more desktop computers, one or mobile devices (e.g., one or more tablets, one or more smartphones, etc.), and/or the like. In non-limiting embodiments, fluid injection system 104 may include one or more injection devices (e.g., one or more fluid injection devices, one or more fluid injectors). In non-limiting embodiments, fluid injection system 104 is configured to administer (e.g., inject, deliver, etc.) contrast fluid including a contrast agent to a patient, and/or administer an aqueous fluid, such as saline, to a patient before, during, and/or after administering the contrast fluid. For example, fluid injection system 104 can be programmed by the user to inject one or more prescribed dosages of contrast fluid directly into a patient's blood stream via a hypodermic needle, syringe or injected by an automated fluid injection system. In some non-limiting embodiments, fluid injection system 104 may be configured to continually administer the aqueous fluid to a patient through a peripheral intravenous line (PIV), or intra-arterially during cardiovascular intervention and one or more prescribed dosages of contrast fluid may be introduced using these aforementioned methods and administered via the catheter to the patient. In some non-limiting embodiments, fluid injection system 104 is configured to inject a dose of contrast fluid along with and/or followed by administration of a particular volume of the aqueous fluid. In non-limiting embodiments, fluid injection system 104 may include one or more exemplary fluid injection devices that are disclosed in: U.S. patent application Ser. No. 09/715,330, filed on Nov. 17, 2000, issued as U.S. Pat. No. 6,643,537; U.S. patent application Ser. No. 09/982,518, filed on Oct. 18, 2001, issued as U.S. Pat. No. 7,094,216; U.S. patent application Ser. No. 10/825,866, filed on Apr. 16, 2004, issued as U.S. Pat. No. 7,556,619; U.S. patent application Ser. No. 12/437,011, filed May 7, 2009, issued as U.S. Pat. No. 8,337,456; U.S. patent application Ser. No. 12/476,513, filed Jun. 2, 2009, issued as U.S. Pat. No. 8,147,464; and U.S. patent application Ser. No. 11/004,670, filed on Dec. 3, 2004, issued as U.S. Pat. No. 8,540,698, the disclosures of each of which are incorporated herein by reference in their entireties. In non-limiting embodiments, fluid injection system 104 may include the MEDRAD® Stellant CT Injection System, the MEDRAD® Stellant FLEX CT Injection System, the MEDRAD® MRXperion MR Injection System, the MEDRAD® Mark 7 Arterion Injection System, the MEDRAD® Intego PET Infusion System, or the MEDRAD® Centargo CT Injection System, all of which are provided by Bayer Healthcare LLC.

In non-limiting embodiments, workstation device 106 may include one or more devices capable of being in communication with event management system 102, fluid injection system 104, user device 108, IoT management system 110, and/or hospital information system 112 via communication network 114. For example, workstation device 106 may include a computing device, such as one or more computers, including a desktop computer, a laptop, tablet, and/or the like. At least a portion of the processes executed at the workstation device 106 may be executed at a remote server (e.g., a cloud computing server). In non-limiting embodiments, workstation device 106 may provide a user interface for controlling operation of fluid injection system 104, including to generate instructions for and/or provide instructions to fluid injection system 104. Additionally, or alternatively, workstation device 106 may display operational parameters of fluid injection system 104 during operation (e.g., during real-time operation) of fluid injection system 104. In non-limiting embodiments, workstation device 106 may provide interconnectivity between fluid injection system 104 and other devices or systems, such as a scanner device (e.g., a Magnetic Resonance Imaging (MRI) scanner, a computed tomography (CT) scanner, a CT coronary angiography system, a Positron Emission Tomography (PET) scanner, a Single Photon Emission Computed Tomography (SPECT) scanner, not shown). In non-limiting embodiments, workstation device 106 may include the Certegra® Workstation provided by Bayer. In non-limiting embodiments, workstation device 106 may include a display unit (e.g., a display device, a display screen, etc. ,), such as a computer monitor, a touchscreen, a heads-up display, and/or the like, which may be used to display a user interface (e.g., a graphical user interface (GUI) of a software application), via which a user may interact with workstation device 106 to view parameters and/or control operation of fluid injection system 104. In some non-limiting embodiments, workstation device 106 may include a speaker for output of tones or sounds related to operation of a device and/or for output of language for communications with the user, for example to affirm or repeat input provided by the user. For example, a user of workstation device 106 may provide inputs to workstation device 106 using one or more hardware or software components of the workstation device 106 in connection with a touch screen, a mouse, a trackpad, a keyboard, a stylus, a gesture-sensing camera, a microphone for receiving voice commands, and/or the like.

In non-limiting embodiments, user device 108 may include one or more devices capable of being in communication with event management system 102, fluid injection system 104, workstation device 106, IoT management system 110, and/or hospital information system 112 via communication network 114. For example, user device 108 may include a computing device, such as one or more computers, including a desktop computer, a workstation device, a mobile device such as a laptop, tablet, smartphone, and/or the like. In non-limiting embodiments, at least a portion of the processes executed at the user device 108 may be executed at a remote server (e.g., a cloud computing server, a server that hosts a software application, etc.). In non-limiting embodiments, user device 108 may provide a user interface for receiving data associated with an event involving a medical device. Additionally, or alternatively, user device 108 may display operational parameters of event management system 102 during operation (e.g., during real-time operation) of event management system 102. In non-limiting embodiments, user device 108 may provide interconnectivity between event management system 102 and other devices or systems, such as fluid injection system 104, workstation device 106, IoT management system 110, and/or hospital information system 112, and/or other systems or devices (e.g., a medical imaging system). In non-limiting embodiments, user device 108 may include a display unit (e.g., a display device, a display screen, etc. ,), such as a computer monitor, a touchscreen, a heads-up display, and/or the like, which may be used to display a user interface (e.g., a graphical user interface (GUI) of a software application), via which a user may interact with user device 108 to view parameters and/or control operation of event management system 102, fluid injection system 104, workstation device 106, IoT management system 110, and/or hospital information system 112. For example, a user of user device 108 may provide inputs to user device 108 using one or more hardware or software components of the user device 108 in connection with a touch screen, a mouse, a trackpad, a keyboard, a stylus, a gesture-sensing camera, a microphone for receiving voice commands, and/or the like.

In non-limiting embodiments, IoT management system 110 may include one or more devices capable of being in communication with event management system 102, fluid injection system 104, workstation device 106, user device 108, and/or hospital information system 112 via communication network 114. For example, IoT management system 110 may include one or more computing devices, such as one or more computers, one or more servers (e.g., a cloud server, a group of servers, etc.), one or more desktop computers, one or more mobile devices (e.g., one or more tablets, one or more smartphones, etc.), and/or the like. In non-limiting embodiments, IoT management system 110 may include one or more (e.g., a plurality of) applications (e.g., software applications) that perform a set of functionalities on an external API that allows IoT management system 110 to send data to an external system associated with the external API and to receive data from the external system associated with the external API. In non-limiting embodiments, the application may be supported by an application associated with user device 108 that would allow IoT management system 110 to be a device that controls other systems and/or devices, and, in such an example, IoT management system 110 may provide an information retrieval function. In non-limiting embodiments, IoT management system 110 may receive data from one or more connected devices (e.g., medical devices) and/or transmit data to one or more connected devices, and the data may include, but is not limited to, protocols, software updates, and/or user access permissions with regard to the connected devices. Other uses may include remote asset monitoring, remote maintenance and/or service, predictive maintenance, asset management, billing, and/or optimized equipment effectiveness. An example of IoT management system 110 may be ThingWorx by PTC Inc. of Boston, MA.

In non-limiting embodiments, hospital information system 112 may include one or more devices capable of being in communication with event management system 102, fluid injection system 104, workstation device 106, user device 108, and/or IoT management system 110 via communication network 114. For example, hospital information system 112 may include one or more computing devices, such as one or more desktop computers, one or more mobile devices, one or more servers, and/or the like. In non-limiting embodiments, hospital information system 112 may include one or more subsystems, such as a patient procedure tracking system (e.g., a system that operates a modality worklist, a system that provides patient demographic information for fluid injection procedures and/or medical imaging procedures, etc.), a fluid injection management system, an image archive and communication system (e.g., a picture archive and communication system (PACS)), a radiology information system (RIS), and/or a radiology analytics system (e.g., the Radimetrics® Enterprise Application marketed and sold by Bayer HealthCare LLC). In non-limiting embodiments, hospital information system 112 may include one or more databases that stores patient records (e.g., data files or data objects that include patient data) therein, which may be accessed (e.g., with read-access permission and/or write-access permission) by devices or systems in the environment 100, such as event management system 102 and/or workstation device 106. In non-limiting embodiments, communication network 114 may include one or more wired and/or wireless networks. For example, communication network 114 may include a cellular network (e.g., a long-term evolution (LTE®) network, a third generation (3G) network, a fourth generation (4G) network, a fifth generation (5G) network, a sixth generation (6G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, a short range wireless communication network (e.g., a Bluetooth® network, a near field communication (NFC) network, etc.) and/or the like, and/or a combination of these or other types of networks.

The number and arrangement of systems and/or devices shown in FIG. 1 are provided as an example. There may be additional systems and/or devices, fewer systems and/or devices, different systems and/or devices, or differently arranged systems and/or devices than those shown in FIG. 1. Furthermore, two or more systems and/or devices shown in FIG. 1 may be implemented within a single system or a single device, or a single system or a single device shown in FIG. 1 may be implemented as multiple, distributed systems or devices. Additionally, or alternatively, a set of systems or a set of devices (e.g., one or more systems, one or more devices) of environment 100 may perform one or more functions described as being performed by another set of systems or another set of devices of environment 100.

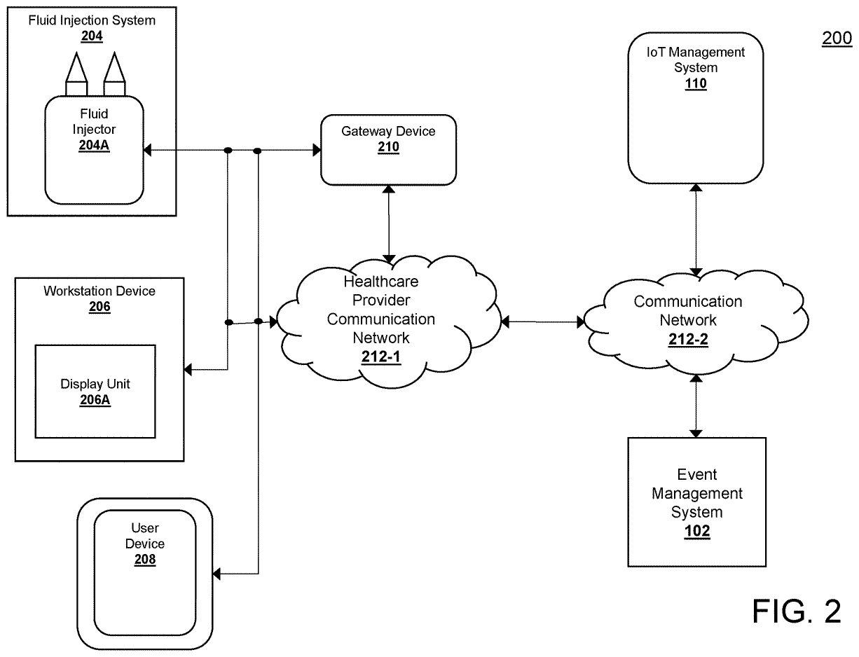

Referring now to FIG. 2, FIG. 2 is a diagram of a non-limiting embodiment of system 200 for processing information associated with an event involving a medical device. In non-limiting embodiments, one or more of the functions described herein with respect to system 200 may be performed (e.g., completely, partially, and/or the like) by event management system 102. In non-limiting embodiments, one or more of the functions described with respect to system 200 may be performed (e.g., completely, partially, and/or the like) by another device or a group of devices separate from and/or including event management system 102, such as fluid injection system 204, workstation device 206, user device 208, and/or IoT management system 110.

As shown in FIG. 2, system 200 includes event management system 102, fluid injection system 204, which includes fluid injector 204A, workstation device 206, which includes display unit 206A, user device 208, gateway device 210, and IoT management system 110. In non-limiting embodiments, event management system 102 may interconnect (e.g., establish a connection to communicate with and/or the like) with fluid injection system 204, workstation device 206, which includes display unit 206A, user device 208, gateway device 210, and/or IoT management system 110 via wired connections, wireless connections, or a combination of wired and wireless connections. In non-limiting embodiments, fluid injection system 204 may be the same as or similar to fluid injection system 104. In non-limiting embodiments, workstation device 206 may be the same as or similar to workstation device 106. In non-limiting embodiments, user device 208 may be the same as or similar to user device 108. In non-limiting embodiments, healthcare provider communication network 212-1 and/or communication network 212-2 may be the same as or similar to communication network 114. In non-limiting embodiments, healthcare provider communication network 212-1 may communicate information according to communication protocols (e.g., a Health Level Seven (HL7) standard communications protocol, a network protocol (e.g., HTTP, HTTPS, etc.), a Digital Imaging and Communications in Medicine (DICOM) communications protocol, etc.) that are appropriate for the setting. In non-limiting embodiments, healthcare provider communication network 212-1 may include a hospital communication network and/or a healthcare enterprise communication network.

In non-limiting embodiments, event management system 102 may include a plurality of applications, and each of the plurality of applications may be associated with an API associated with a respective application (e.g., a first API associated with a first application, a second API associated with a second application, a third API associated with a third application, etc.) that allows other systems and/or devices to interface (e.g., communicate, establish a communication interface, etc.) with event management system 102 and/or that allows event management system 102 to interface with other systems and/or devices (e.g., individual subsystems of hospital information system 112) via healthcare provider communication network 212-1 and communication network 212-2. In non-limiting embodiments, event management system 102 may provide a user interface (e.g., via an application that includes a user interface, such as a web-based user interface) that allows a user to access, receive, store, and/or transmit information. For example, event management system 102 may provide a user interface that is displayed on fluid injection system 204, user device 206, and/or workstation device 206 (e.g., display unit 206A of workstation device 206).

As further shown in FIG. 2, workstation device 206 may include display unit 206A. In non-limiting embodiments, display unit 206A may be capable of displaying the user interface (e.g., the web-based user interface) provided by event management system 102. In non-limiting embodiments, display unit 206A may include a computing device, such as a smart display unit, a portable computer, such as a tablet, a laptop, and/or the like. In non-limiting embodiments, display unit 206A may include a touchscreen for receiving inputs by a user. In non-limiting embodiments, display unit 206A may include a display device (e.g., a monitor, a screen, and/or the like for displaying visual information) or a speaker for communicating information through sound or language.

In non-limiting embodiments, gateway device 210 may include one or more devices (e.g., one or more communication gateways) capable of being in communication with event management system 102, fluid injection system 204, workstation device 206, user device 208, and/or IoT management system 110 via healthcare provider communication network 212-1 and/or communication network 212-2. For example, gateway device 210 may include a telecommunications gateway (e.g., a network gateway), a universal gateway, and/or other like devices. Additionally, or alternatively, gateway device 210 may include one or more computing devices, such as one or more desktop computers, one or mobile devices (e.g., one or more tablets, one or more smartphones, etc.), one or more servers, and/or the like. In non-limiting embodiments, gateway device 210 may include one or more (e.g., a plurality of) applications (e.g., software applications) that perform a set of functionalities on an external application programming interface (API) that allows fluid injection system 204, workstation device 206, and/or user device 208 to send data to an external system associated with the external API and to receive data from the external system associated with the external API. In one example, gateway device 210 may provide the ability to download an application, such as a Bayer S/W application, to gateway device 210 (e.g., a mobile device of gateway device 210, which provides a Bring Your Own Device operability). In non-limiting embodiments, the application may be supported by an application associated with fluid injection system 204 that would allow gateway device 210 (e.g., a mobile device of gateway device 210), which may function as a control room display, to be the only one device that controls fluid injection system 204 and, in such an example, gateway device 210 may provide an authentication function. In non-limiting embodiments, gateway device 210 may be able to access a network resource, such as a uniform resource locator (URL), to determine applications (e.g., services of applications) that are available to gateway device 210 and gateway device 210 and/or fluid injection system 104 may download one or more applications via gateway device 210. Further details regarding gateway device 210 and associated systems and/or devices are disclosed in International Patent Application No. PCT/US2021/040800, filed on Jul. 8, 2021, which published as WO 2022/015560 on Jan. 20, 2022, the disclosure of which is incorporated herein by reference in its entirety.

In non-limiting embodiments, event management system 102 may communicate with fluid injection system 204, workstation device 206, and/or user device 208 via healthcare provider communication network 212-1 and communication network 212-2, such that fluid injection system 204, workstation device 206, and/or user device 208 are able to receive data based on an API call to event management system 102. In non-limiting embodiments, event management system 102 may transmit data associated with informatics (e.g., data associated with an identification characteristic of a medical device received from IoT management system 110) to fluid injection system 204 via a communication network (e.g., healthcare provider communication network 212-1 and communication network 212-2). For example, event management system 102 may transmit data associated with informatics received from IoT management system 110 to fluid injection system 204 via the communication network based on an API call from fluid injection system 204.

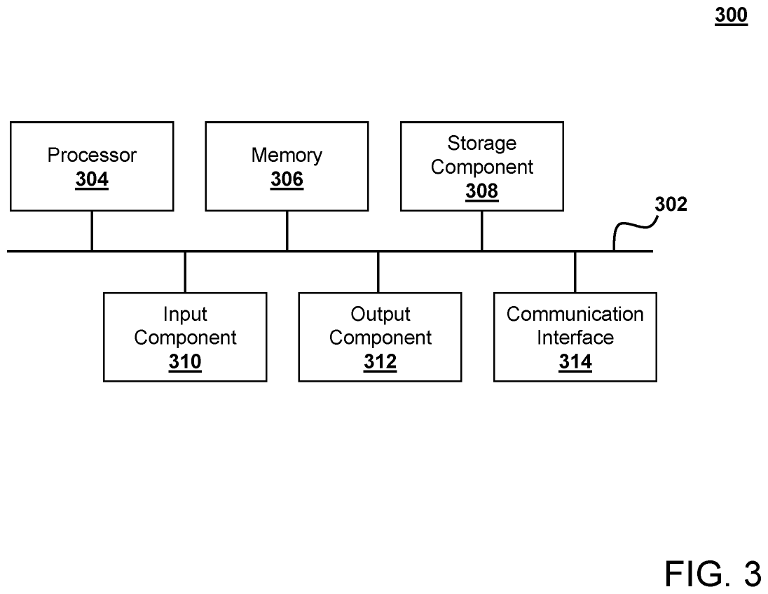

Referring now to FIG. 3, FIG. 3 is a diagram of example components of device 300. Device 300 may correspond to one or more devices of event management system 102, fluid injection system 104, fluid injection system 204, workstation device 106, workstation device 206, user device 108, user device 208, IoT management system 110, gateway device 210, and/or hospital information system 112. In non-limiting embodiments, event management system 102, fluid injection system 104, fluid injection system 204, workstation device 106, workstation device 206, user device 108, user device 208, IoT management system 110, gateway device 210, and/or hospital information system 112 may include at least one device 300 and/or at least one component of device 300.

As shown in FIG. 3, device 300 may include bus 302, processor 304, memory 306, storage component 308, input component 310, output component 312, and communication interface 314. Bus 302 may include a component that permits communication among the components of device 300. In non-limiting embodiments, processor 304 may be implemented in hardware, firmware, or a combination of hardware and software. For example, processor 304 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 306 may include random access memory (RAM), read only memory (ROM), and/or another type of dynamic or static storage device (e.g., flash memory, magnetic memory, optical memory, etc.) that stores information and/or instructions for use by processor 304.

Storage component 308 may store information and/or software related to the operation and use of device 300. For example, storage component 308 may include a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 310 may include a component that permits device 300 to receive information, such as via user input (e.g., a touch screen display, a keyboard, a keypad, a mouse, a button, a switch, a microphone, etc.). Additionally, or alternatively, input component 310 may include a sensor for sensing information (e.g., a global positioning system (GPS) component, an accelerometer, a gyroscope, an actuator, etc.). Output component 312 may include a component that provides output information from device 300 (e.g., a display, a speaker, one or more light-emitting diodes (LEDs), etc.).

Communication interface 314 may include a transceiver-like component (e.g., a transceiver, a separate receiver and transmitter, etc.) that enables device 300 to communicate with other devices, such as via a wired connection, a wireless connection, or a combination of wired and wireless connections. Communication interface 314 may permit device 300 to receive information from another device and/or provide information to another device. For example, communication interface 314 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 300 may perform one or more processes described herein. Device 300 may perform these processes based on processor 304 executing software instructions stored by a computer-readable medium, such as memory 306 and/or storage component 308. A computer-readable medium (e.g., a non-transitory computer-readable medium) is defined herein as a non-transitory memory device. A memory device may include memory space located inside of a single physical storage device or memory space spread across multiple physical storage devices.

Software instructions may be read into memory 306 and/or storage component 308 from another computer-readable medium or from another device via communication interface 314. When executed, software instructions stored in memory 306 and/or storage component 308 may cause processor 304 to perform one or more processes described herein. Additionally, or alternatively, hardwired circuitry may be used in place of or in combination with software instructions to perform one or more processes described herein. Thus, embodiments described herein are not limited to any specific combination of hardware circuitry and software.

The number and arrangement of components shown in FIG. 3 are provided as an example. In non-limiting embodiments, device 300 may include additional components, fewer components, different components, or differently arranged components than those shown in FIG. 3. Additionally, or alternatively, a set of components (e.g., one or more components) of device 300 may perform one or more functions described as being performed by another set of components of device 300.

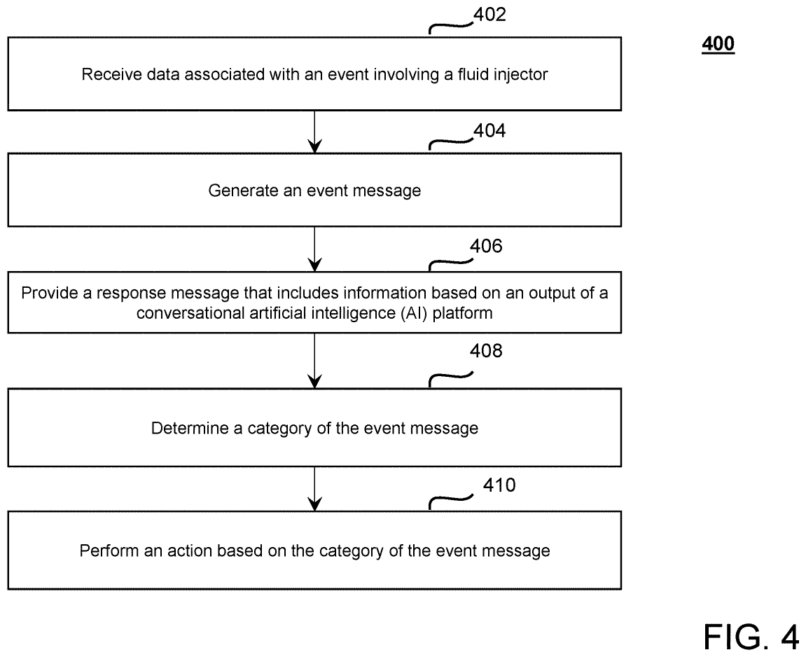

Referring now to FIG. 4, FIG. 4 is a flowchart of a non-limiting embodiment of a process 400 for processing information associated with an event involving a medical device. In non-limiting embodiments, one or more of the steps of process 400 are performed (e.g., completely, partially, etc.) by event management system 102. In non-limiting embodiments, one or more of the steps of process 400 are performed (e.g., completely, partially, etc.) by another device or a group of devices separate from or including event management system 102, such as a fluid injection system (e.g., fluid injection system 104, such as one or more devices of fluid injection system 104), a workstation device (e.g., workstation device 106), a user device (e.g., user device 108), an IoT management system (e.g., IoT management system 110, such as one or more devices of IoT management system 110), and/or a hospital information system (e.g., hospital information system 112, such as one or more subsystems of hospital information system 112). According to non-limiting embodiments, while FIG. 4 is described in the context of an event involving a medical device, the steps of process 400 may be performed with regard to another type of medical device, such as a medical imaging device, a surgical device, a health monitoring device, and/or the like.

As shown in FIG. 4, at step 402, process 400 may include receiving data associated with an event involving a medical device. For example, event management system 102 may receive data associated with an event involving a medical device of fluid injection system 204. In non-limiting embodiments, event management system 102 may receive the data via a user interface (UI) associated with the medical device of fluid injection system 204. For example, event management system 102 may receive the data via a UI displayed on display unit 206A of workstation device 206, displayed on user device 208, displayed on a display screen of fluid injection system 204, and/or the like. In non-limiting embodiments, the UI may be a UI of a software application (e.g., a mobile application, a client-side application of a web-based software application, etc.) located on workstation device 206, user device 208, and/or fluid injection system 204.

In non-limiting embodiments, event management system 102 may receive the data associated with the event involving the medical device via a generative and conversational AI platform. For example, event management system 102 may receive the data via a generative AI model, a chatbot, or any combination thereof. In non-limiting embodiments, event management system 102 may receive the data via a chatbot and/or generative AI model that interacts with a UI associated with the medical device. For example, a user may provide an input to a chatbot and/or generative AI model via a UI on user device 108, and event management system 102 may receive the data as an output of a the chatbot and/or the generative AI model. In non-limiting embodiments, event management system 102 may receive voice data from a user via the UI associated with the medical device, convert the voice data to text data using a speech to text algorithm, and convert the text data to the data associated with the event involving the medical device using a natural language processing (NLP) algorithm. In non-limiting embodiments, event management system 102 may receive the data associated with the event involving the medical device via an email, a text, and/or as data from an interaction with a UI (e.g., a selection of graphical user element of a UI, a button press, etc.).

In non-limiting embodiments, event management system 102 may receive data associated with an identification characteristic of the medical device of fluid injection system 204. For example, event management system 102 may receive data associated with an identification characteristic of the medical device from IoT management system 110. In non-limiting embodiments, IoT management system 110 may provide the data associated with the identification characteristic of the medical device based on receiving a notification of an event involving the medical device. For example, IoT management system 110 may provide the data associated with the identification characteristic of the medical device based on receiving the notification from event management system 102, fluid injection system 204, workstation device 206, and/or user device 208 via healthcare provider communication network 212-1 and/or communication network 212-2.

In non-limiting embodiments, event management system 102 may provide the UI associated with the medical device based on receiving an input prompt (e.g., a voice prompt) by a mobile device. For example, event management system 102 may provide the UI associated with the medical device on fluid injection system 204 (e.g., a fluid injector on fluid injection system 204), workstation device 206, and/or user device 208, via an application (e.g., a software application) of fluid injection system 204, workstation device 206, and/or user device 208 based on receiving the input prompt. In non-limiting embodiments, event management system 102 may receive the data associated with the event involving the medical device before, during, and/or after a fluid injection procedure involving the medical device (e.g., a fluid injector).

In non-limiting embodiments, event management system 102 may provide the input prompt to receive information from a user via the UI associated with the medical device. In non-limiting embodiments, the input prompt includes one or more questions associated with the event involving the medical device. For example, the input prompt may include a question asking for a description of the event involving the medical device. Additionally, or alternatively, the input prompt may include a series of successive questions (e.g., “YES” or “NO”) associated with a type of event involving the medical device. In non-limiting embodiments, event management system 102 may provide one or more questions asking for a description of the event involving the medical device, where the one or more questions are pre-scripted or generated by the generative and conversational AI platform based on the data associated with the event involving the medical device received from a user (e.g., a user of fluid injection system 204, workstation device 206, and/or user device 208).

In non-limiting embodiments, event management system 102 may provide one or more response prompts based on receiving initial data associated with the event involving the medical device. For example, event management system 102 may receive initial data associated with the event involving the medical device and event management system 102 may provide one or more response prompts that request additional data associated with the event involving the medical device (e.g., data in addition to the initial data associated with the event involving the medical device for an event message).

In non-limiting embodiments, event management system 102 may receive the data associated with the event involving the medical device via healthcare provider communication network 212-1 and/or communication network 212-2. For example, a user may provide the data associated with the event involving the medical device to a UI displayed on display unit 206A of workstation device 206, displayed on user device 208, and/or displayed on a display screen of fluid injection system 204, and the data may be provided to gateway device 210. Gateway device 210 may transmit the data to event management system 102 via healthcare provider communication network 212-1 and communication network 212-2.

In non-limiting embodiments, the event may include an incident related to an adverse event (e.g., a fluid injection procedure that involves a patient safety event, including extravasation, over volume of contrast media, an air injection, particulate in a fluid path, electrocution, and/or any other safety related event) for the medical device. Additionally, or alternatively, the event may include a malfunction of the medical device and/or a safety issue, an event related to an issue with a pharmaceutical product (e.g., a contrast agent for a fluid injector), and/or an event related to a product quality issue. In non-limiting embodiments, a product quality issue may relate to a need to service for a medical device, a product replacement or a replacement of a component of a product, a clinical application of a medical device, or a customer complaint concerning a system (e.g., a fluid injector including associated disposables).

In non-limiting embodiments, the data associated with an event involving a medical device may include an indication of a type of event involving the medical device. For example, the data associated with the event involving the medical device may include an indication of an event related to an adverse event for the medical device, an indication of an event related to an issue with a pharmaceutical product of a medical device, and/or indication of an event related to a product quality issue. Additionally, or alternatively, the data associated with the event involving the medical device may include data associated with a time stamp regarding the event involving the medical device. In non-limiting embodiments, the data associated with the time stamp may include data associated with a time at which the data associated with the event involving the medical device was received (e.g., from a user via the UI associated with the medical device) and/or data associated with a time at which an issue (e.g., a malfunction) of the medical device occurred. In non-limiting embodiments, event management system 102 may capture data associated with a time stamp regarding the event involving the medical device.

Additionally, or alternatively, the data associated with an event involving a medical device may include product details related to the event involving the medical device including an injector model identification, a serial number of the medical device, a lot number of disposables used in a fluid injection procedure, an amount (e.g., a volume) of contrast agent used in a fluid injection procedure, and/or an amount of contrast agent and/or flushing agent (e.g., saline) delivered to a patient in a fluid injection procedure.

Additionally, or alternatively, the data associated with an event involving a medical device may include exam details related to the event involving the medical device such as injection parameters, including a size of a catheter used in a fluid injection procedure, timing, flow rate and/or volume of a fluid used in a fluid injection procedure, a pressure setting used in a fluid injection procedure, components in a fluid path used in a fluid injection procedure, a type of scan performed in association with a fluid injection procedure, whether a personalized software program of an injection protocol was used in a fluid injection procedure, a location at which a fluid injection procedure was initiated, an indication as to whether an injection site was monitored (e.g., visually monitored) during a fluid injection procedure, an indication of whether a change in a pressure monitor occurred during a fluid injection procedure, and/or a type of scan being performed with regard to a fluid injection procedure.

Additionally, or alternatively, the data associated with an event involving a medical device may include extravasation details related to the event involving the medical device such as when extravasation occurred (e.g., start, middle, or end) during a fluid injection procedure, a location of an affected area on a body of a patient where an extravasation occurred, an amount of extravasated fluid involved in an extravasation, an amount of extravasated or tissue infiltrated fluid involved in an extravasation, a type of fluid (e.g., contrast and/or saline) involved in an extravasation, a pressure setting during a fluid injection procedure in which an extravasation occurred, and/or an indication as to whether scanner bolus detection software was used during a fluid injection procedure in which an extravasation occurred.

Additionally, or alternatively, the data associated with an event involving a medical device may include air injection details related to the event involving the medical device such as a location of air in a body (e.g., coronary vessels, pulmonary trunk or branches, etc.) of a patient, an amount of air (e.g., a minor amount, 5 ml, 10ml, an amount larger than a threshold amount, etc.), an indication of whether a prime tube was used, and/or an indication of whether a wet-to-wet connection was used when preparing disposable.

Additionally, or alternatively, the data associated with an event involving a medical device may include particulate in fluid path details related to the event involving the medical device such as an indication of whether a specific type of disposables were used, an indication of when a particulate was noticed (e.g., an indication of when opening a kit, an indication of during use, an indication of following a procedure, etc.), and/or a lot number of one or more disposables that were impacted.

Additionally, or alternatively, the data associated with an event involving a medical device may include patient outcome details related to the event involving the medical device such as age of a patient, gender of a patient, admitting diagnosis of a patient, pre-existing conditions of a patient, an indication of whether a patient required any type of medical treatment (e.g., intervention) during and/or after an event, an indication of a type of medical treatment provided to a patient during and/or after an event if treatment was provided, an indication of a condition of a patient during and/or after an event if treatment was or was not provided, and/or an indication of a reason as to why a procedure was ordered or being performed (e.g., an indication of what a physician was hoping to rule out).

Additionally, or alternatively, the data associated with an event involving a medical device may include complaint details related to the event involving the medical device such as an image of a product, an image of a defect of a product, an identification number of a product and/or a part of a product that malfunctioned, and/or a description of a malfunction (e.g., failure) of a product, such as a method of malfunction (e.g., a product fell), an indication of when a product was last serviced, an indication of who provided service to a product when the product was last serviced, and/or an indication of whether there was a noticeable issue (e.g., noticeable defect, difficulty in operation, etc.) with a product prior to a malfunction. Additionally, or alternatively, the data associated with an event involving a medical device may include product outcome details such as an indication of a result of a malfunction of a product, a description of an adverse outcome based on a malfunction of a product, an indication of whether a patient required any type of medical treatment as a result of a malfunction of a product, an indication of a safety allegation with regard to a product, a description of an adverse outcome based on a safety allegation of a product, and/or an indication of whether a patient required any type of medical treatment as a result of a safety allegation of a product.

As shown in FIG. 4, at step 404, process 400 may include generating an event message. For example, event management system 102 may generate the event message based on the data associated with the event involving the medical device. Additionally, or alternatively, event management system 102 may generate the event message based on data associated with the identification characteristic of the medical device. In non-limiting embodiments, the event message may include the data associated with the event involving the medical device and/or the data associated with the identification characteristic of the medical device.

As shown in FIG. 4, at step 406, process 400 may include providing a response message that includes information based on an output of a generative and conversational artificial intelligence (AI) platform. For example, event management system 102 may provide one or more response messages that includes information based on an output of the generative and conversational AI platform. In non-limiting embodiments, the generative and conversational AI platform includes at least one of a generative AI model, a chatbot, or any combination thereof.

In non-limiting embodiments, the one or more response messages may include the output of the generative and conversational AI platform. In non-limiting embodiments, the one or more response messages may include information associated with the output of the generative and conversational AI platform, such as a question that requests data associated with an event involving a medical device of fluid injection system 204. In non-limiting embodiments, event management system 102 may provide the response message via a UI associated with the medical device (e.g., a UI displayed on fluid injection system 204, workstation device 206, and/or user device 208) based on the data associated with the event involving the medical device.

In non-limiting embodiments, event management system 102 may generate a response message based on receiving the data associated with the event involving the medical device. In one example, event management system 102 may provide data associated with the event involving the medical device as an input to a generative AI model and event management system 102 may determine an output of the generative AI model based on the input. The output may include information associated with a potential resolution of the event involving the medical device. In non-limiting embodiments, event management system 102 may provide the information associated with the potential resolution of the event involving the medical device. For example, event management system 102 may provide the information associated with the potential resolution of the event involving the medical device to fluid injection system 204, workstation device 206, and/or user device 208 (e.g., via a UI associated with the medical device).

In non-limiting embodiments, event management system 102 may provide one or more response messages based on receiving the data associated with the event involving the medical device. In non-limiting embodiments, the one or more response messages may include one or more response questions (e.g., “YES” or “NO”) that determine an action to be performed by event management system 102. In one example, a response question may include a question as to whether a system checkout is desired. If an affirmative response is received, event management system 102 may determine to transmit an event message to a customer complaints system and/or a product service system. In one example, a response question may include a question as to whether an appointment is desired. If an affirmative response is received, event management system 102 may determine to transmit an event message to a customer complaints system and/or a clinical applications system. In non-limiting embodiments, a response question may include one or more questions generated by the generative and conversational AI platform based on data associated with the event involving the medical device. In non-limiting embodiments, a response question may be based on an output of a generative and conversational AI platform.

As shown in FIG. 4, at step 408, process 400 may include determining a category of the event message. In non-limiting embodiments, event management system 102 may determine a category of the event message based on at least one of the data associated with the event involving the medical device, the data associated with the identification characteristic of the medical device, or any combination thereof. In non-limiting embodiments, the category of the event message may be a category associated with a system to which event management system 102 may transmit the event message. In non-limiting embodiments, the category may be a category associated with a complaint system, a service system (e.g., a service scheduling system), a hospital information system (e.g., hospital information system 112), a regulatory reporting system, and/or the like. In non-limiting embodiments, event management system 102 may determine a category of the event message from a plurality of categories of an event message.

In non-limiting embodiments, event management system 102 may determine a category of the event message based on an output of a machine learning model. For example, event management system 102 may provide the data associated with the event involving the medical device and/or the data associated with the identification characteristic of the medical device as an input to the machine learning model, and the machine learning model may provide an output based on the input, where the output includes a prediction of a category of the event message.

In non-limiting embodiments, event management system 102 may determine a category of the event message based on an indication (e.g., an indication associated with a type of event) included in the data associated with the event involving the medical device. For example, event management system 102 may receive the data associated with the event involving the medical device and event management system 102 may determine an indication of a category of the event message included in the data associated with the event involving the medical device. event management system 102 may determine the category based on the indication of the category of the event message.

Referring again to FIG. 4, process 400 is described above as including, in step 408, determining a category of an event message. The category may be further described, in non-limiting embodiments, as being a category associated with a complaint system, a service system (e.g., a service scheduling system), a hospital information system (e.g., hospital information system 112), a regulatory reporting system, and/or the like. Examples of such a complaint system may include a complaint system associated with a manufacturer of a medical device such as a fluid injector (e.g., Bayer Medical Care Inc.), a complaint system associated with a regulatory body (e.g., the U.S. Food and Drug Administration (USFDA), the European Medicines Agency (EMA), the Pharmaceuticals and Medical Devices Agency (PhMDA) of Japan, the Medicines and Healthcare products Regulatory Agency (MHRA) of the United Kingdom), and/or the like. Examples of such a service system may include a service system associated with a manufacturer of a medical device such as a fluid injector (e.g., Technical Assistance Center (TAC) of Bayer Medical Care, Inc.), a service system associated with a third-party under contract to a manufacturer of a medical device, and/or the like. Examples of such a regulatory reporting system may include the Adverse Event Reporting System (FAERS) of the USFDA, the Medical Devices Vigilance System of the European Union (EU), the EudraVigilance system of the EMA, and/or the like.

As shown in FIG. 4, at step 410, process 400 may include performing an action based on the category of the event message. For example, event management system 102 may perform an action based on the category of the event message. In non-limiting embodiments, event management system 102 transmit, store (e.g., record), and/or analyze the event message based on the category of the event message.

In non-limiting embodiments, event management system 102 may transmit the event message to a healthcare provider communication network, a hospital information system (e.g., hospital information system 112), a system for trend analysis with regard to one or more events involving the medical device, a system associated with a regulatory body, a communication gateway for further transmission, and/or the like.

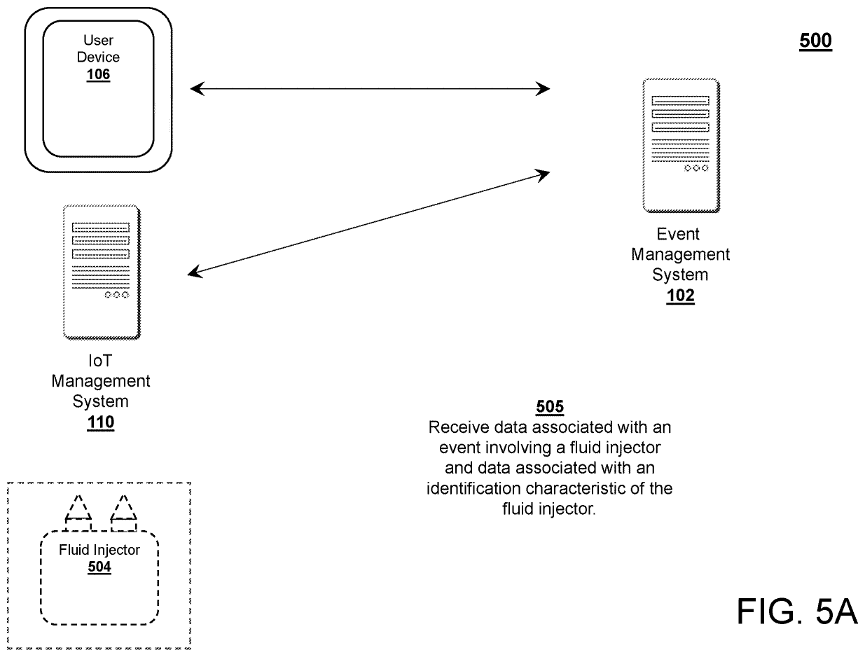

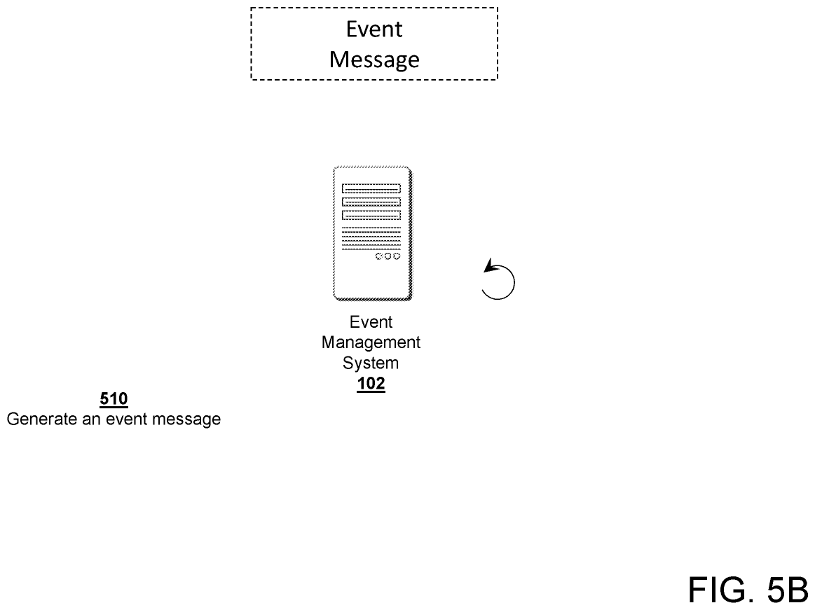

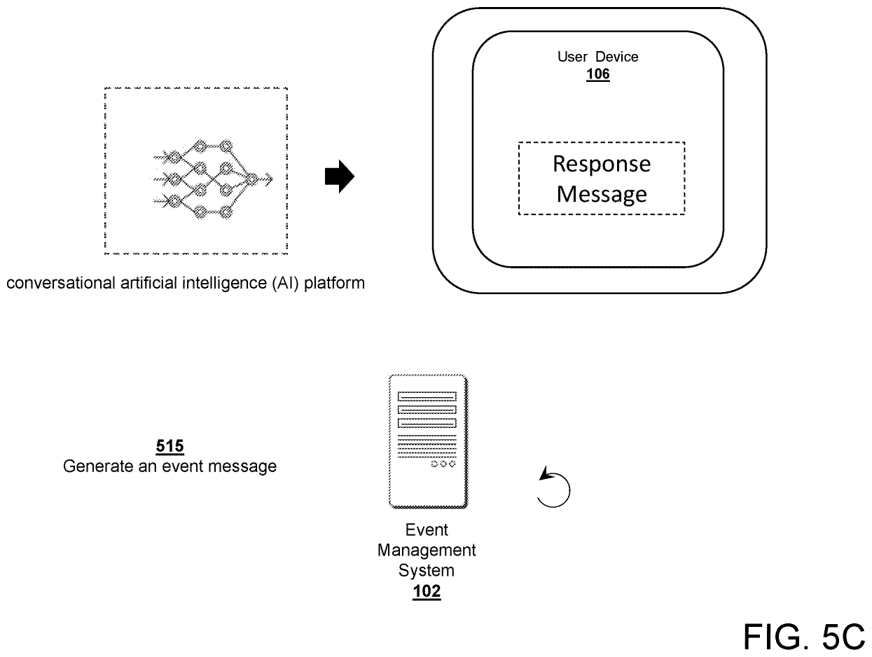

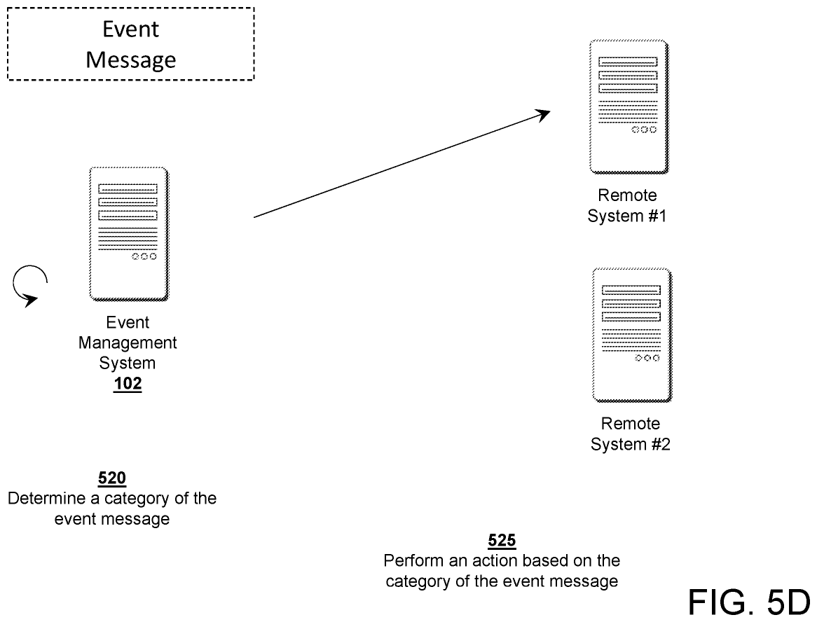

Referring now to FIGS. 5A-5D, FIGS. 5A-5D are diagrams of a non-limiting embodiment or aspect of implementation 500 relating to a process (e.g., process 400) for event management for a medical device, such as fluid injector 504. In some non-limiting embodiments, fluid injector 504 may be the same as or similar to fluid injection system 104, fluid injection system 204, and/or fluid injector 204A. In non-limiting embodiments or aspects, one or more of the steps of the process may be performed (e.g., completely, partially, etc.) by event management system 102 (e.g., by one or more devices of event management system 102). In non-limiting embodiments or aspects, one or more of the steps of the process may be performed (e.g., completely, partially, etc.) by another device or a group of devices separate from or including event management system 102, such as a fluid injection system (e.g., fluid injection system 104, such as one or more devices of fluid injection system 104), a workstation device (e.g., workstation device 106), a user device (e.g., user device 108), an IoT management system (e.g., IoT management system 110, such as one or more devices of IoT management system 110), and/or a hospital information system (e.g., hospital information system 112, such as one or more subsystems of hospital information system 112).

As shown by reference number 505 in FIG. 5A, event management system 102 may receive data associated with an event involving fluid injector 504 and data associated with an identification characteristic of fluid injector 504. For example, event management system 102 may receive the data associated with the event involving fluid injector 504 from user device 108 and event management system 102 may receive the data associated with an identification characteristic of fluid injector 504 from IoT management system 110. In non-limiting embodiments, event management system 102 may receive the data associated with the event involving fluid injector 504 via a UI associated with fluid injector 504 displayed on user device 208. In non-limiting embodiments, IoT management system 110 may provide the data associated with the identification characteristic of fluid injector 504 to event management system 102 based on receiving a notification of the event involving fluid injector 504.

As shown by reference number 510 in FIG. 5B, event management system 102 may generate an event message. In non-limiting embodiments, event management system 102 may generate the event message based on the data associated with the event involving fluid injector 504 and the data associated with the identification characteristic of fluid injector 504. In non-limiting embodiments, the event message may include the data associated with the event involving fluid injector 504 and the data associated with the identification characteristic of fluid injector 504.

As shown by reference number 515 in FIG. 5C, event management system 102 may provide a response message. In non-limiting embodiments, event management system 102 may provide the response message via the UI displayed on user device 108. In non-limiting embodiments, the response message may include information based on an output of a generative and conversational AI platform. In one example, event management system 102 may generate the response message that includes information based on an output of a generative and conversational AI platform. In non-limiting embodiments, the generative and conversational AI platform includes at least one of a generative AI model, a machine learning model, a conversational AI model, such as a chatbot, or any combination thereof.

As shown by reference number 520 in FIG. 5D, event management system 102 may determine a category of the event message. In non-limiting embodiments, event management system 102 may determine the category of the event message based on at least one of the data associated with the event involving fluid injector 504, the data associated with the identification characteristic of fluid injector 504, or any combination thereof. In non-limiting embodiments, the category of the event message may be a category associated with a remote system to which event management system 102 may transmit the event message. In non-limiting embodiments, event management system 102 may determine a category of the event message from a plurality of categories of an event message.

As shown by reference number 525 in FIG. 5D, event management system 102 may perform an action based on the category of the event message. In non-limiting embodiments, event management system 102 may determine a remote system of a plurality of remote systems to which to transmit the event message based on the category of the event message. In non-limiting embodiments, event management system 102 may transmit the event message to a first remote system (e.g. remote system #1) based on the category of the event message. Examples of such a remote system may include a remote service system associated with a manufacturer of a medical device such as a fluid injector (e.g., VirtualCARE® Remote Support of Bayer Medical Care, Inc, Technical Assistance Center (TAC) of Bayer Medical Care, Inc.), a remote service system associated with a third-party under contract to a manufacturer of a medical device, and/or the like.

Although the above systems, methods, and computer program products have been described in detail for the purpose of illustration based on what is currently considered to be the most practical and preferred embodiments, it is to be understood that such detail is solely for that purpose and that the present disclosure is not limited to the described embodiments or aspects but, on the contrary, is intended to cover modifications and equivalent arrangements that are within the spirit and scope of the appended claims. For example, it is to be understood that the present disclosure contemplates that, to the extent possible, at least one feature of any embodiment or aspect can be combined with at least one feature of any other embodiment.

The following non-limiting example scenarios make use of an event management system (e.g., event management system 102) as described herein:
Scenario 1—A service issue. During a patient MR exam the technologist notices a burning smell around the injector and scanner. She discontinues the exam and presses the alert button on the workstation GUI in the control room. The workstation pops open an entry field and prompts the tech what the reason is for her call. The tech is able to immediately and verbally describe that there is a burning smell in the scan room while she was filling the injector with fluids. Once the tech stops speaking, the system responds by saying that the serial number of the injector and scanner combination was sent to the Bayer service desk and has alerted a service tech to come out within xx hours. The system responds with a warning to not use the injector until a Bayer service tech has inspected it.
Scenario 2—A complaint—A technologist complains that the volume on the workstation is way too loud and wants to connect with someone to remedy the situation. She presses the alert button on the workstation GUI in the control room. The workstation pops open an entry field and prompts the tech what the reason is for her call. The tech is able to immediately and verbally describe that the noise coming from the workstation when making a selection is very loud and annoying. Once the tech stops speaking, the system responds by saying that the serial number of the injector and scanner combination was sent to the Bayer Technical Assistance Center (TAC) for further review and management.
Scenario 3—An adverse event—A Brazilian site reports an incident from a mobile CT unit and requests an injector checkout following an alleged air injection. The CT technologist presses the alert button on the workstation GUI in the control room. The workstation pops open an entry field and prompts the tech what the reason is for her call. The tech is able to immediately and verbally inform of the air injection and that there was a resulting artifact in the diagnostic scan and wants to request an injector checkout. Once the tech stops speaking, the system responds audio/in writing that the event report has been de-identified and shared with the hospital admin for review and the request for injector checkout is forwarded (serial number of the injector and scanner combination) to the Bayer Service and Adverse Events Team (Issue & Complaint Management (ICM) team). Someone from the Complaints team will contact the customer to gather event details and to dispatch service to the site. The system responds with a link to a digital resource that contains a brief video how to ensure no air is in the patient line.
Scenario 4—A complaint—A technologist complains that when filling a syringe using a spike, excessive leaking ensues. This is causing delay as additional time is needed to clean up the spill. The workstation pops open an entry field and prompts the tech what the reason is for her call. The tech is able to immediately and verbally request assistance. Once the tech stops speaking, the system responds by saying that the serial number of the injector and scanner combination was sent to a TAC representative. The TAC representative triages the information and determines that the customer needs to touch base with the Clinical Performance Team to adjust the auto load purges volume setting on the unit.
Scenario 5—A complaint—Diagnostic imaging artifacts. A customer sees some ringing in an MR scan and wonders if it is caused by the MR injector. The person calls or enters the complaint on the injector. Either through a text chat or voice communications, the system listens and asks diagnostic questions. Based upon the responses the system recommends “debugging” steps to the customer, and depending upon the results recommends the next step. If necessary, it provides access to human assistance.
Scenario 6—An adverse event—The customer reported an alleged extravasation during an enhanced CT procedure while a patient was connected to a MEDRAD® Stellant CT Injection System. The technologist requests an injector checkout to ensure proper operation. The user presses the alert button on the workstation GUI in the control room. The workstation opens an entry field and prompts the user to input the reason for the call. The technologist is able to immediately and verbally inform Bayer of the extravasation and would like to request a system service check by Bayer field service. Once the caller stops speaking, the system responds with a notification that the event report has been identified and the request for the injector checkout is forwarded to Bayer Service to dispatch to field service and the Issue & Complaint Management (ICM) team. A member from ICM will follow-up with the customer to gather additional event related details. The system also sends a request to the Clinical Performance Center for clinical applications follow-up.
Scenario 7—A complaint—Connectivity issues with an integrated system caused the injector to abort and the scanner had to be restarted and there is an exam interruption. There was a dose administered and the patient needs to be rescanned. The system may look into a data base and, if injector-scanner interface (ISI) problem(s) are well known with this system configuration, inform the user what steps should be taken to remedy the situation. If this is not a known problem, then via textual chat or voice communications the system may go through a series of questions and replies with the customer to help debug the system. If a solution is found, this exchange will be added to the data base for reference in subsequent cases. If a solution is not found, the system may recommend contacting the imager manufacture and may put in a service alert to the Bayer service center to make them aware of the problem and that a service call may be requested.
Scenario 8—Request for information—“I need help with P3T protocol. I haven't used it in months and this procedure requires it.” is a verbal message transmitted to the system through the injector user interface. The system responds by connecting the user to training videos online and the appropriate section in the manual as text. These types of requests for information may all be handled on the injector or on the tech's phone via a custom QR code that injector displays.
Scenario 9—Request for help—“I need to order something” is stated verbally or otherwise into the injector user interface. The system responds by displaying a triage menu to ask specifically what is needed. Once a specific thing or things are selected, the system connects the user directly to Bayer customer care for ordering for more disposables. If the request is for a new injector, the system emails this request to the applicable salesperson with a confirmation going to the user If the request is for a replacement syringe heater, the communication goes to Bayer service for shipment of the part or scheduling of a visit, depending on the service contract that is in place. If the categorization of the request is “other”, the user is connected to Bayer customer care.
Scenario 10—Request for help—“My injector doesn't work. I need service.” is entered into the system by one of the user interfaces. The system looks at the service agreement that is in place and either contacts Bayer service if the service of that equipment is currently covered by Bayer or, if the service is handled by the local hospital biomed or a 3^{rd} party service agency, the system will contact the appropriate number, email, or website to transfer the request for service.
Scenario 11—A request that leads to a Professional Inquiry Request (PIR) Customer has a question that relates to one or more of the following subjects which our commercial people cannot handle, for example the off-label use of contrast agent or a device. The user speaks their PIR into the system. The system looks to see if an acceptable answer is available. If so, it shares that answer with the user, including references. It then checks to see if the user is satisfied. If no acceptable answer is available or the user is not satisfied with the answer given, then it forwards the request to appropriate medical or R&D person(s) for human follow up.

In all Scenarios, the system may ask the user for their satisfaction with answers given or actions taken and for their interactions with the system. This information may be used to refine future responses and to identify gaps in knowledge which may need to be added to the system.

1. A system for processing information associated with an event involving a medical device, comprising:
at least one processor configured to:
receive data associated with an event involving a medical device via a user interface (UI) associated with the medical device;
receive data associated with an identification characteristic of the medical device;
generate an event message, wherein the event message comprises the data associated with the event involving the medical device and the data associated with the identification characteristic of the medical device;
provide a response message via the UI associated with the medical device based on the data associated with the event involving the medical device, wherein the response message comprises information based on an output of a generative and conversational artificial intelligence (AI) platform;
determine a category of the event message based on at least one of the data associated with the event involving the medical device, the data associated with the identification characteristic of the medical device, or any combination thereof; and
perform an action based on the category of the event message.

2. The system of claim 1, wherein the generative and conversational AI platform comprises at least one of a generative AI model, a conversational AI model, or any combination thereof.

3. The system of claim 1, wherein the at least one processor is configured to:
generate the response message based on receiving the data associated with the event involving the medical device, wherein, when generating the response message, the at least one processor is configured to:
provide the data associated with the event involving the medical device as an input to a generative AI model;
determine an output of the generative AI model based on the input, wherein the output comprises information associated with a potential resolution of the event involving the medical device;
wherein, when providing the response message via the UI associated with the medical device, the at least one processor is configured to:
provide the information associated with the potential resolution of the event involving the medical device.

4. The system of claim 1, wherein, when providing the response message via the UI associated with the medical device, the at least one processor is configured to:
provide a prompt to receive information from a user via the UI associated with the medical device, wherein the prompt comprises a question associated with the event involving the medical device.

5. The system of claim 1, wherein, when performing the action, the at least one processor is configured to:
transmit the event message based on the category of the event message.

6. The system of claim 5, wherein, when transmitting the event message, the at least one processor is configured to:
transmit the event message to a healthcare provider communication network;
transmit the event message to a system for trend analysis with regard to one or more events involving the medical device;
transmit the event message to a communication gateway for further transmission; or
any combination thereof.

7. The system of claim 1, wherein the at least one processor is further configured to:
capture data associated with a time stamp regarding the event involving the medical device, wherein the data associated with the time stamp comprises at least one of:
data associated with a time at which the data associated with the event involving the medical device was received via the UI associated with the medical device;
data associated with a time at which a malfunction of the medical device occurred; or
any combination thereof.

8. The system of claim 1, wherein the at least one processor is further configured to:
provide the UI associated with the medical device on a mobile device.

9. The system of claim 8, wherein, when providing the UI associated with the medical device on the mobile device, the at least one processor is configured to:
provide the UI associated with the medical device on the mobile device via an application of the mobile device based on receiving an input prompt at the mobile device.

10. The system of claim 1, wherein, when receiving the data associated with the event involving the medical device, the at least one processor is configured to:
receive the data associated with the event involving the medical device via a chatbot that interacts with the UI associated with the medical device.

11. The system of claim 1, wherein, when receiving the data associated with the event involving the medical device, the at least one processor is configured to:
receive voice data from a user via the user interface (UI) associated with the medical device;
convert the voice data to text data using a speech to text algorithm; and
convert the text data to the data associated with the event involving the medical device using a natural language processing (NLP) algorithm.

12-22. (canceled)

23. A computer program product for processing information associated with an event involving a medical device, comprising at least one non-transitory computer-readable medium including one or more instructions, that when executed by at least one processor cause the at least one processor to:
receive data associated with an event involving a medical device via a user interface (UI) associated with the medical device;
receive data associated with an identification characteristic of the medical device;
generate an event message, wherein the event message comprises the data associated with the event involving the medical device and the data associated with the identification characteristic of the medical device;
provide a response message via the UI associated with the medical device based on the data associated with the event involving the medical device, wherein the response message comprises information based on an output of a generative and conversational artificial intelligence (AI) platform;
determine a category of the event message based on at least one of the data associated with the event involving the medical device, the data associated with the identification characteristic of the medical device, or any combination thereof; and
perform an action based on the category of the event message.

24. The computer program product of claim 23, wherein the generative and conversational AI platform comprises at least one of a generative AI model, a conversational AI model, or any combination thereof.

25. The computer program product of claim 23, wherein the one or more instructions further cause the at least one processor to:
generate the response message based on receiving the data associated with the event involving the medical device, wherein, the one or more instructions that cause the at least one processor to generate the response message cause the at least one processor to:
provide the data associated with the event involving the medical device as an input to a generative AI model;
determine an output of the generative AI model based on the input, wherein the output comprises information associated with a potential resolution of the event involving the medical device;
wherein, the one or more instructions that cause the at least one processor to provide the response message via the UI associated with the medical device cause the at least one processor to:
provide the information associated with the potential resolution of the event involving the medical device.

26. The computer program product of claim 23, wherein, the one or more instructions that cause the at least one processor to provide the response message via the UI associated with the medical device cause the at least one processor to:
provide a prompt to receive information from a user via the UI associated with the medical device, wherein the prompt comprises a question associated with the event involving the medical device.

27. The computer program product of claim 23, wherein, the one or more instructions that cause the at least one processor to perform the action cause the at least one processor to:
transmit the event message based on the category of the event message.

28. The computer program product of claim 27, wherein, the one or more instructions that cause the at least one processor to transmit the event message cause the at least one processor to:
transmit the event message to a healthcare provider communication network;
transmit the event message to a system for trend analysis with regard to one or more events involving the medical device;
transmit the event message to a communication gateway for further transmission; or
any combination thereof.

29. The computer program product of claim 23, wherein the one or more instructions further cause the at least one processor to:
capture data associated with a time stamp regarding the event involving the medical device, wherein the data associated with the time stamp comprises at least one of:
data associated with a time at which the data associated with the event involving the medical device was received via the UI associated with the medical device;
data associated with a time at which a malfunction of the medical device occurred; or
any combination thereof.

30. The computer program product of claim 23, wherein the one or more instructions further cause the at least one processor to:
provide the UI associated with the medical device on a mobile device.

31. The computer program product of claim 30, wherein, the one or more instructions that cause the at least one processor to provide the UI associated with the medical device on the mobile device cause the at least one processor to:
provide the UI associated with the medical device on the mobile device via an application of the mobile device based on receiving an input prompt at the mobile device.

32. The computer program product of claim 23, wherein, the one or more instructions that cause the at least one processor to receive the data associated with the event involving the medical device cause the at least one processor to:
receive the data associated with the event involving the medical device via a chatbot that interacts with the UI associated with the medical device.

33. The computer program product of claim 23, wherein, when receiving the data associated with the event involving the medical device, the at least one processor is configured to:
receive voice data from a user via the user interface (UI) associated with the medical device;
convert the voice data to text data using a speech to text algorithm; and
convert the text data to the data associated with the event involving the medical device using a natural language processing (NLP) algorithm.
